(19) United States
(12) Patent Application Publication
(10) Pub. No.: US 2008/0213218 A1
(43) Pub. Date: Sep. 4, 2008

(54) METHOD FOR TREATING HEPATITIS C VIRUS INFECTION IN TREATMENT FAILURE PATIENTS

(75) Inventor: Henry H. Hsu, (US)

Correspondence Address: ARENT FOX LLP, 1050 CONNECTICUT AVENUE, N.W., SUITE 400, WASHINGTON, DC, 20036, US

(73) Assignee: VALEANT PHARMACEUTICALS INTERNATIONAL, INC., Aliso Viejo, CA (US)

(21) Appl. No.: 12/034,542

(22) Filed: Feb. 20, 2008

Related U.S. Application Data
(63) Continuation of application No. 10/490,456, filed on Mar. 22, 2004, now abandoned.
(60) Provisional application No. 60/326,100, filed on Sep. 28, 2001.

(51) Int. Cl. A61K 38/21; A61P 31/14

(52) U.S. Cl. 424/85.7

(57) ABSTRACT

The present invention provides methods for treating individuals having a hepatitis C virus (HCV) infection, which individuals have failed to respond to therapy with an IFN-α other than consensus interferon (CIFN), or who, following cessation of therapy with an IFN-α other than CIFN, have suffered relaspe. The methods generally involve a treatment regimen comprising administering a first dosing regimen of CIFN, followed by a second dosing regimen of CIFN. Ribavirin is administered with at least the second dosing regimen.

### FIELD OF THE INVENTION

This invention is in the field of treating viral infections, and in particular, treating hepatitis C virus infection.

### BACKGROUND OF THE INVENTION

Hepatitis C virus (HCV) infection is the most common chronic blood borne infection in the United States. Although the numbers of new infections have declined, the burden of chronic infection is substantial, with Centers for Disease Control estimates of 3.9 million (1.8%) infected persons in the United States. Chronic liver disease is the tenth leading cause of death among adults in the United States, and accounts for approximately 25,000 deaths annually, or approximately 1% of all deaths. Studies indicate that 40% of chronic liver disease is HCV-related, resulting in an estimated 8,000-10,000 deaths each year. HCV-associated end-stage liver disease is the most frequent indication for liver transplantation among adults.

The high prevalence of chronic HCV infection has important public health implications for the future burden of chronic liver disease in the United States. Data derived from the National Health and Nutrition Examination Survey (NHANES III) indicate that a large increase in the rate of new HCV infections occurred from the late 1960s to the early 1980s, particularly among persons between 20 to 40 years of age. It is estimated that the number of persons with long-standing HCV infection of 20 years or longer could more than quadruple from 1990 to 2015, from 750,000 to over 3 million. The proportional increase in persons infected for 30 or 40 years would be even greater. Since the risk of HCV-related chronic liver disease is related to the duration of infection, with the risk of cirrhosis progressively increasing for persons infected for longer than 20 years, this will result in a substantial increase in cirrhosis-related morbidity and mortality among patients infected between the years of 1965-1985.

Antiviral therapy of chronic hepatitis C has evolved rapidly over the last decade, with significant improvements seen in the efficacy of treatment. Nevertheless, even with combination therapy using pegylated IFN-α plus ribavirin, 40% to 50% of patients fail therapy. These patients are generally referred to as “treatment failure” patients, and include both non-responders (patients in whom viral titer remains high even during therapy) and relapsers (patients in whom viral titers drop initially during therapy, but subsequently rise either during therapy or after treatment has ended). These patients currently have no effective therapeutic alternative. In particular, patients who have advanced fibrosis or cirrhosis on liver biopsy are at significant risk of developing complications of advanced liver disease, including ascites, jaundice, variceal bleeding, encephalopathy, and progressive liver failure, as well as a markedly increased risk of hepatocellular carcinoma.

Type I interferons are cytokines that exhibit both antiviral and antiproliferative activity. Type I interferons include interferon-α (IFN-α) and interferon-β IFN-α includes naturally occurring IFN-α, and derivatives having the amino acid sequence of a naturally occurring IFN-α, such as in PEGylated IFN-α. Naturally occurring IFN-α that have been used in anti-viral therapies includes IFN-α2a, IFN-αa2b. Derivatives of naturally occurring IFN-α, e.g., PEGylated IFN-α's, have also been used in antiviral therapy.

Consensus IFN-α's (IFN-con; IFN alfacon; CIFN) are forms of non-naturally occurring type I IFN-α. Consensus interferon alphas include IFN-con₁, IFN-con₂, and IFN-con₃. In vitro studies comparing the relative antiviral, antiproliferative, and natural killer cell activities of recombinant CIFN with either leukocyte or other recombinant type-one interferons demonstrate that CIFN displays significantly higher activity when compared on a mass basis. Others have reported that CIFN, when used in the treatment of diseases susceptible to treatment by alpha interferons, does not cause the same degree of side effects in patients as do the alpha interferons. It has also been reported that 3 to 5 times higher doses of CIFN can be used, leading to enhanced therapeutic benefit, with substantially no corresponding increase in the frequency or severity of undesirable side effects. Some success has been reported in the use of CIFN monotherapy to treat patients that failed to respond to IFN-α therapy.

Even in view of the therapies currently available, there remains a need for improved therapies for treatment failure patients. The present invention addresses this need.

Literature

U.S. Pat. No. 5,980,884. U.S. Pat. No. 5,372,808. Aliaga, S. et al., Farmacia Clinica (Spain) 14(5):324-331 (June 1997); Bailly, F. et al., Nephrol. Dial. Transplant. 11(Suppl. 4):56-57 (1996); Bizollon, T. et al., Hepatol. 26:500-504 (1997); Brillanti, S. et al., J. Hepatol. 23(Suppl. 2):13-16 (1995); Camps, J. et al., J. Hepatol. 19:408-412 (1993); Davis et al., Hepatol. 26(Suppl. 1):122S-127S (September 1997); Davis, G. L., Gastroenterol. Clin. N. Amer. 23(3):603-613 (1994); Dusheiko, G. M. et al., Br. Med. J. 312:357-364 (1996); Fried, M. W., Med. Clin. N. Amer. 80(5):957-972 (1996); Lindsay, K., Hepatol. 26(Suppl. 1):71S-77S (September 1997); Mazzaferro, V. et al., Transplant. Prop. 29:519-521 (1997); McHutchison, J., Hepatol. 26(2):505-506 (August 1997); Merican, M. I., Med. J. Malaysia 47(3):158-169 (1992); Poupon, R. and Serfaty, L., Bull. Acad. Natle. Med. 180(6):1279-1289 (1996); Reichard, O., Scand. J. Infect. Dis. (Suppl. 95):1-56 (1994); Saracco, G. and Rizzetto, M., Drugs 53(1):74-85 (1997); Schalm, S. W. and Brouwer, J. T., Scand. J. Gastroenterol. 223:46-49 (1997); Schalm, S. W. et al., Dig. Dis. Sci. 41(12): 131S-134S (December 1996); Scotto, G. et al., Ital. J. Gastroenterol. 28:505-511 (1996); Scotto, G. et al., J. Chemother. 7(1):58-61 (1995); Theodor, E. and Regev, A., Harefuah 132(6):402-403; 447 (1997); Thomas, H. C. et al., Drugs 52(Suppl. 2):1-8 (1996); Tillmann, H. and Manns, M., Kidney Blood Press. Res. 19(3-4):215-219 (1996); Tong, M. et al., J. Gastroenterol. Hepatol. 9:587-591 (1994); Trepo, C. et al., Nephrol. Dial. Transplant. 11(Suppl. 4):62-64 (1996); Weiss, R. and Oostrom-Ram, T., Vet. Microbiol. 20:255-265 (1989); Chemello, L. et al., J. Hepatol. 23(Suppl. 2):8-12 (1995); Main, J., J. Hepatol. 23(Suppl. 2):32-36 (1995); Schalm, S. W. et al., J. Hepatol. 26:961-966 (May 1997); Sherlock, S., J. Hepatol. 23(Suppl. 2):3-7 (1995); Braconier, J. et al., Scand. J. Infect. Dis. 27:325-329 (1995); Brillanti, S. et al., Gastroenterol. 107:812-817 (1994); Chemello, L. et al., J. Hepatol. 21(Suppl. 1):s12 Abstract No. GS 5/29 (1994); Cohen, J., Science 285:26-30 (2 Jul. 1999); Lai, M-Y. et al., Gastroenterol. 111:1307-1312 (1996); McHutchison, J. G. et al., N. Eng. J. Med. 339(21):1485-1491 (1998); Poynard, T. et al., The Lancet 352(9138):1426-1432 (1998; Schvarcz, R. et al., J. Hepatol. 23(Suppl. 2):17-21 (1995); and Schvarcz, R. et al., J. Med. Virol. 46(1):43-47 (1995)

Melian and Plosker (2001) Drugs 61:1-31; Heathcote et al. (1998) Hepatol. 27:1136-1143; Heathcote et al. (1999) Hepatol. 30:562-566; Sjögren et al. (Apr. 30, 2000) 35^{th}Annual Meeting of the European Association for the Study of the Liver Rotterdam; Chow et al. (1998) Hepatol. 27:1144-1148; Chemello et al. (1997) C. Gastroenterol. 113:1654-1659; Davis et al. (1998) N. Engl. J. Med. 339:1493-1499; Kaiser et al. (Apr. 20, 2001) 36^{th} Annual Meeting of the European Association for the Study of the Liver, Prague; Sjögren (Apr. 20, 2001) 36^{th} Annual Meeting of the European Association for the Study of the Liver, Prague.

### SUMMARY OF THE INVENTION

The present invention provides methods for treating individuals having a hepatitis C virus (HCV) infection, which individuals have failed to respond to therapy with an IFN-α other than consensus interferon (CIFN), or who, following cessation of therapy with an IFN-α other than CIFN, have suffered relapse. The methods generally involve a treatment regimen comprising administering a first dosing regimen of CIFN, followed by a second dosing regimen of CIFN. Ribavirin is administered with at least the second dosing regimen.

In one aspect, the invention features a method for treating a hepatitis C virus infection in an individual. The methods generally involve delivery of CIFN and ribavirin, where CIFN is administered in a therapeutic regimen comprising a first dosing regimen of CIFN, followed by a second dosing regimen of CIFN, where the lowest average daily serum concentration of CIFN achieved by the first dosing regimen is greater than the highest average daily serum concentration of CIFN achieved by the second dosing regimen. Ribavirin is administered during administration of at least the last dosing event of the second dosing regimen, and may be administered with additional dosing events continuous with the last dosing event during which ribavirin is administered. The individual treated has failed previous IFN-α-based therapy, e.g., the individual has either failed to respond to IFN-α therapy other than CIFN therapy, or, following cessation of IFN-α therapy other than CIFN therapy, has suffered a relapse.

### DEFINITIONS

The term “treatment failure patients” (or “treatment failures”) as used herein generally refers to HCV-infected patients who failed to respond to previous therapy for HCV (referred to as “non-responders”) or who initially responded to previous therapy (e.g., in whom an initial viral response (IVR) was observed), but in whom the therapeutic response was not maintained (referred to as “relapsers”). The previous therapy generally can include treatment with IFN-α monotherapy, or IFN-α combination therapy, where the IFN-α combination therapy may include administration of IFN-α and an antiviral agent such as ribavirin.

The terms “non-CIFN IFN-α therapy,” and “IFN-α therapy other than CIFN,” as used interchangeably herein in the context of previous IFN-α therapy, refer to any IFN-α-based therapy, other than therapy that includes administration of CIFN, including IFN-α monotherapy and IFN-α combination therapy (e.g., IFN-α and an antiviral such as ribavirin).

The terms “non-CIFN IFN-α” and “IFN-α other than CIFN,” used interchangeably herein, refer to IFN-α that is not consensus CIFN and includes, but is not limited to, IFN-α2a; IFN-α2b; IFN-α2C; recombinant forms of naturally-occurring IFN-α, mixtures of naturally occurring IFN-α (e.g., IFN-αn1 and IFN-αn3); and derivatives, e.g., PEGylated derivatives, of the foregoing. The term specifically excludes consensus IFN-α, as defined below.

The term “consensus IFN-α” (used interchangeably herein with “CIFN” and “IFN-alpha con”), as used herein refers specifically to a synthetic interferons including IFN-con₁, IFN-con₂, IFN-con₃, and derivatives thereof, e.g., PEGylated derivatives. PEGylated derivatives of CIFN can be produced according to methods in the art (see, e.g., U.S. Pat. Nos. 5,985,265; 5,382,657; 5,559,213; and 6,177,074).

The term “early viral response,” used interchangeably with “initial viral response” (“IVR”) refers to the drop in viral titer within about 24 hours, about 48 hours, about 2 days, or about 1 week after the beginning of treatment for HCV infection.

The term “sustained viral response” (SVR; also referred to as a “sustained response” or a “durable response”), as used herein, refers to the response of an individual to a treatment regimen for HCV infection, in terms of serum HCV titer. Generally, a “sustained viral response” refers to no detectable HCV RNA (e.g., less than about 500, less than about 200, or less than about 100 genome copies per milliliter serum) found in the patient's serum for a period of at least about one month, at least about two months, at least about three months, at least about four months, at least about five months, or at least about six months following cessation of treatment.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease (as in liver fibrosis that can result in the context of chronic HCV infection); (b) inhibiting the disease, i.e., arresting its development; and (c) relieving the disease, i.e., causing regression of the disease.

The terms “individual,” “host,” “subject,” and “patient” are used interchangeably herein, and refer to a mammal, including, but not limited to, primates, including simians and humans, with humans being of particular interest.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a dose” includes a plurality of such doses and reference to “the method” includes reference to one or more methods and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

The present invention provides methods of treating hepatitis C virus (HCV) infection in individuals having an HCV infection and have failed treatment, e.g., individuals who have failed to respond to IFN-α therapy other than consensus interferon (CIFN) therapy; or who, during or following cessation of IFN-α therapy other than CIFN therapy, have suffered a relapse. The methods generally involve administration of CIFN and an antiviral agent such as ribavirin as follows: 1) administering a first dosing regimen of CIFN, optionally with a dosing regimen of ribavirin; 2) followed by a second dosing regimen of CIFN and a dosing regimen of ribavirin. The lowest average daily serum concentration of CIFN achieved by the first dosing regimen is higher than the highest average daily serum concentration of CIFN achieved by the second dosing regimen. The first and second dosing regimen of ribavirin may be the same or different.

The first dosing regimen of CIFN (also referred to as “the induction regimen”) generally involves administration of CIFN at about 9 μg, about 15 μg, about 18 μg, or about 27 μg. The first dosing regimen can encompass a single dosing event, or at least two or more dosing events.

The first dosing regimen of CIFN can be administered daily, every other day, three times a week, or substantially continuously so as to achieve a desired average daily serum concentration of CIFN.

The first dosing regimen of CIFN (which may be administered in combination with an antiviral such as ribavirin) is administered for a first period of time, which time period can be at least about 4 weeks, at least about 8 weeks, or at least about 12 weeks.

The first dosing regimen of CIFN (optionally administered with ribavirin) is effective to reduce viral titer to a low viral titer, e.g., a reduction of at least about 0.5 log, at least about 1.0 log, at least about 1.5 log, at least about 2.0 log, at least about 2.5 log, at least about 3.0 log, at least about 3.5 log, at least about 4.0 log, at least about 4.5 log, or at least about 5 log, compared to the pre-treatment viral titer, is achieved by the end of the first dosing regimen.

The second dosing regimen of CIFN (also referred to as “the maintenance dose”) generally involves administration of at least about 3 μg, at least about 9 μg, at least about 15 μg, or at least about 18 μg of CIFN. The second dosing regimen can encompass a single dosing event, or at least two or more dosing events.

The second dosing regimen of CIFN can be administered daily, every other day, three times a week, or substantially continuously so as to achieve a desired average daily serum concentration of CIFN.

The second dosing regimen of CIFN (in combination with ribavirin) is effective to reduce viral titer still further, e.g., to undetectable levels, e.g., to from about 500 genome copies per ml serum, to less than or about 200 genome copies per ml serum, or to less than or about 100 genome copies per ml serum.

The second dosing regimen of CIFN is administered for at least about 8 weeks, at least about 12 weeks, at least about 20 weeks, at least about 24 weeks, or at least about 48 weeks.

The treatment regimen described above (i.e., the first and second dosing regimens) effects a durable response (also referred to as a “sustained response”), e.g., no detectable HCV RNA is found in the patient's serum for a period of at least about one month, at least about two months, at least about three months, at least about four months, at least about five months, or at least about six months following cessation of a treatment regimen as described herein.

CIFN is administered in combination with an antiviral agent. The antiviral agent can be administered simultaneously in separate formulations; simultaneously in the same formulation; administered in separate formulations and within about 48 hours, within about 36 hours, within about 24 hours, within about 16 hours, within about 12 hours, within about 8 hours, within about 4 hours, within about 2 hours, within about 1 hour, within about 30 minutes, or within about 15 minutes or less. Where the CIFN and the antiviral agent are delivered as separate formulations, the CIFN and the antiviral agent may be delivered by the same or different routes. The antiviral agent may be delivered in the same or different dosing regimen as the CIFN.

In one embodiment, patients are treated with a combination of CIFN and ribavirin. Ribavirin, 1-β-D-ribofuranosyl-1H-1,2,4-triazole-3-carboxamide, available from ICN Pharmaceuticals, Inc., Costa Mesa, Calif., is described in the Merck Index, compound No. 8199, Eleventh Edition. Its manufacture and formulation is described in U.S. Pat. No. 4,211,771. The invention also contemplates use of derivatives of ribavirin (see, e.g., U.S. Pat. No. 6,277,830). The ribavirin may be administered orally in capsule or tablet form, or in the same or different administration form and in the same or different route as the CIFN. Of course, other types of administration of both medicaments, as they become available are contemplated, such as by nasal spray, transdermally, by suppository, by sustained release dosage form, etc. Any form of administration will work so long as the proper dosages are delivered without destroying the active ingredient.

Ribavirin is generally administered in an amount ranging from about 30 mg to about 60 mg, from about 60 mg to about 125 mg, from about 125 mg to about 200 mg, from about 200 mg to about 300 gm, from about 300 mg to about 400 mg, from about 400 mg to about 1200 mg, from about 600 mg to about 1000 mg, or from about 700 to about 900 mg per day.

In some embodiments, ribavirin is administered throughout the entire course of CIFN therapy. Ribavirin is administered with at least the last dosing regimen, and may be administered with the last dosing regimen and any additional dosing regimen within the treatment regimen continuous with the last dosing regimen. For example, where the treatment regimen includes four dosing events, ribavirin is administered with the fourth dose, and may optionally be administered with the third and fourth doses, the second, third, and fourth doses, or with the first, second, third and fourth doses.

Exemplary, non-limiting treatment regimens include the following.

Treatment Regimen 1A: 15 μg CIFN/day for eight weeks, followed by 9 μg CIFN/day for 16 weeks to 40 weeks. Ribavirin is administered 1000-1200 mg per day throughout the treatment regimen.

Treatment Regimen 1B: 15 μg CIFN/day for eight weeks, followed by 9 μg CIFN/day for 16 weeks to 40 weeks. Ribavirin is administered 1000-1200 mg per day for the last 16-40 weeks.

Treatment Regimen 2A: 15 μg CIFN/day for eight weeks, followed by 15 μg CIFN three times per week (TIW) for 16-40 weeks. Ribavirin is administered 1000-1200 mg per day throughout the treatment regimen.

Treatment Regimen 2B: 15 μg CIFN/day for eight weeks, followed by 15 μg CIFN three times per week (TIW) for 16-40 weeks. Ribavirin is administered 1000-1200 mg per day for the last 16-40 weeks.

Treatment Regimen 3A: 27 μg CIFN/day for four weeks, followed by 18 μg CIFN/day for eight weeks, followed by 9 μg CIFN day for 12 weeks, followed by 9 μg CIFN TIW for 24 weeks. Ribavirin is administered 1000-1200 mg per day throughout the treatment regimen.

Treatment Regimen 3B: 27 μg CIFN/day for four weeks, followed by 18 μg CIFN/day for eight weeks, followed by 9 μg CIFN day for 12 weeks, followed by 9 μg CIFN TIW for 24 weeks Ribavirin is administered 1000-1200 mg per day beginning with the eight week course of 18 μg CIFN/day and continued for the remainder of the treatment regimen.

Treatment Regimen 3C: 27 μg CIFN/day for four weeks, followed by 18 μg CIFN/day for eight weeks, followed by 9 μg CIFN/day for 12 weeks, followed by 9 μg CIFN TIW for 24 weeks Ribavirin is administered 1000-1200 mg per day beginning with the 12 week course of 9 μg CIFN/day and continued for the remainder of the treatment regimen.

Treatment Regimen 3D: 27 μg CIFN/day for four weeks, followed by 18 μg CIFN/day for eight weeks, followed by 9 μg CIFN day for 12 weeks, followed by 9 μg CIFN TIW for 24 weeks Ribavirin is administered 1000-1200 mg per day beginning with the 24 week course of 9 μg CIFN/TIW and continued for the remainder of the treatment regimen.

Treatment Regimen 4A: 18 μg CIFN/day for four weeks, followed by 9 μg CIFN/day for 20 weeks, followed by 9 μg CIFN TIW for 24 weeks. Ribavirin is administered 1000-1200 mg per day throughout the treatment regimen.

Treatment Regimen 4B: 18 μg CIFN/day for four weeks, followed by 9 μg CIFN/day for 20 weeks, followed by 9 μg CIFN TIW for 24 weeks. Ribavirin is administered 1000-1200 mg per day beginning with the 20 week course of 9 μg CIFN/day and continued throughout the treatment regimen.

Treatment Regimen 4C: 18 μg CIFN/day for four weeks, followed by 9 μg CIFN/day for 20 weeks, followed by 9 μg CIFN TIW for 24 weeks. Ribavirin is administered 1000-1200 mg per day beginning with the 24 week course of 9 μg CIFN TIW and continued throughout the treatment regimen.

Treatment Regimen 5A: 9 μg CIFN/day for 8-12 weeks, followed by 9 μg CIFN three times a week for the balance of the treatment period (e.g., 36 to 40 weeks), wherein the treatment period is a total of 48 weeks. Ribavirin is administered 1000-1200 mg per day throughout the treatment regimen.

Treatment Regimen 5B: 9 μg CIFN/day for 8-12 weeks, followed by 9 μg CIFN three times a week (TIW) for the balance of the treatment period (e.g., 36 to 40 weeks), wherein the treatment period is a total of 48 weeks. Ribavirin is administered 1000-1200 mg per day beginning with administration of the treatment course of 9 μg CIFN three times a week and continued throughout the remainder of the treatment regimen.

Guidance for dosage regimens is found in the art. See, e.g., Kaiser et al. (Apr. 20, 2001) 36^{th} Annual Meeting of the European Association for the Study of the Liver, Prague; Sjögren (Apr. 20, 2001) 36^{th} Annual Meeting of the European Association for the Study of Association for the Study of the Liver, Rotterdam; and Balmon Melian and Plosker (2001) Drugs 61:1-31; and U.S. Pat. No. 5,980,884.

### IFN-Alpha

The instant methods involve administering to a “treatment-failure” patient an amount of CIFN and ribavirin effective to reduce viral titer and to effect a sustained viral response. Treatment failure patients include non-responders and relapsers who previously underwent treatment with IFN-α other than CIFN. Such previous treatments include treatment with non-CIFN IFN-α monotherapy, and non-CIFN IFN-α combination therapy (e.g., non-CIFN IFN-α plus ribavirin).

The term “non CIFN interferon-alpha” as used herein refers to IFN-α proteins, other than CIFN, that inhibit viral replication and cellular proliferation and modulate immune response. The term “non CIFN interferon-alpha” includes: (1) any naturally occurring IFN-α; (2) recombinant interferon alpha-2b such as Intron-A interferon available from Schering Corporation, Kenilworth, N.J.; (3) recombinant interferon alpha-2a such as Roferon interferon available from Hoffmann-La Roche, Nutley, N.J.; (4) recombinant interferon alpha-2C such as Berofor alpha 2 interferon available from Boehringer Ingelheim Pharmaceutical, Inc., Ridgefield, Conn.; (5) interferon alpha-n1, a purified blend of natural alpha interferons such as Sumiferon available from Sumitomo, Japan or as Wellferon interferon alpha-n1 (INS) available from the Glaxo-Wellcome Ltd., London, Great Britain; (6) interferon alpha-n3 a mixture of natural alpha interferons made by Interferon Sciences and available from the Purdue Frederick Co., Norwalk, Conn., under the Alferon Tradename.

The term “non-CIFN IFN-α” also encompasses derivatives of non-CIFN IFN-α that are derivatized to alter certain properties such as serum half-life. As such, the term “non-CIFN IFN-α” includes glycosylated non-CIFN IFN-α; non-CIFN IFN-α derivatized with polyethylene glycol (“PEGylated IFN-α”); and the like. PEGylated IFN-α, and methods for making same, are discussed in, e.g., U.S. Pat. Nos. 5,382,657; 5,981,709; 5,824,784; 5,985,265; and 5,951,974. PEGylated IFN-α encompasses conjugates of PEG and any of the above-described IFN-α molecules, including, but not limited to, PEG conjugated to interferon alpha-2a (Roferon, Hoffman La-Roche, Nutley, N.J.), interferon alpha 2b (Intron, Schering-Plough, Madison, N.J.), interferon alpha-2c (Berofor Alpha, Boehringer Ingelheim, Ingelheim, Germany).

The term “consensus IFN-α” (also referred to as “CIFN” and “IFN-con”) includes CIFN such as those described in U.S. Pat. Nos. 4,897,471 and 4,695,623 (e.g., Examples 7, 8 or 9 thereof) and the specific product available from Amgen, Inc., (Infergen®, Amgen, Thousand Oaks, Calif.). The term encompasses but is not limited to the amino acid sequences designated IFN-con₁, IFN-con₂ and IFN-con₃ which are disclosed in U.S. Pat. Nos. 4,695,623 and 4,897,471. DNA sequences encoding IFN-con can be synthesized as described in the aforementioned patents or other standard methods.

### Additional Therapeutic Agents

CIFN therapy according to the invention can be carried out in conjunction with therapy for diseases and disorders other than HCV that an individual having an HCV may suffer from. Such diseases include human immunodeficiency virus (HTV) infection; disorders include disorders associated with HTV infection, and include, but are not limited to, fungal infections, respiratory tract infections, infections of the eye, Kaposi's sarcoma, and the like.

CIFN can be administered together with (i.e., simultaneously in separate formulations; simultaneously in the same formulation; administered in separate formulations and within about 48 hours, within about 36 hours, within about 24 hours, within about 16 hours, within about 12 hours, within about 8 hours, within about 4 hours, within about 2 hours, within about 1 hour, within about 30 minutes, or within about 15 minutes or less) one or more additional therapeutic agents. Therapeutic agents that can be administered in combination therapy include, but are not limited to, anti-inflammatory, anti-viral, anti-fungal, anti-mycobacterial, antibiotic, amoebicidal, trichomonocidal, analgesic, anti-neoplastic, anti-hypertensives, anti-microbial and/or steroid drugs.

In some embodiments, patients are treated with a combination of IFN-α and one or more of the following; beta-lactam antibiotics, tetracyclines, chloramphenicol, neomycin, gramicidin, bacitracin, sulfonamides, nitrofurazone, nalidixic acid, cortisone, hydrocortisone, betamethasone, dexamethasone, fluocortolone, prednisolone, triamcinolone, indomethacin, sulindac, acyclovir, amantadine, rimantadine, recombinant soluble CD4 (rsCD4), anti-receptor antibodies (e.g., for rhinoviruses), nevirapine, cidofovir (Vistide™), trisodium phosphonoformate (Foscarnet™), famcyclovir, pencyclovir, valacyclovir, nucleic acid/replication inhibitors, zidovudine (AZT, Retrovir™), didanosine (dideoxyinosine, ddI, Videx™), stavudine (d4T, Zerit™), zalcitabine (dideoxycytosine, ddC, Hivid™), nevirapine (Viramune™), lamivudine (Epivir™, 3TC), protease inhibitors, saquinavir (Invirase™, Fortovase™), ritonavir (Norvir™), nelfinavir (Viracept™), efavirenz (Sustiva™), abacavir (Ziagen™), amprenavir (Agenerase™) indinavir (Crixivan™), ganciclovir, AzDU, delavirdine (Rescriptor™), kaletra, trizivir, rifampin, clathiromycin, erythropoietin, colony stimulating factors (G-CSF and GM-CSF), non-nucleoside reverse transcriptase inhibitors, nucleoside inhibitors, adriamycin, fluorouracil, methotrexate, asparaginase and combinations thereof.

Formulations and Routes of Administration

CIFN and ribavirin are generally administered to individuals in a formulation (e.g., in the same or in separate formulations) with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al, eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The therapeutic agents CIFN and ribavirin, as well as additional therapeutic agents as described herein for combination therapies, can be administered orally, subcutaneously, intramuscularly, parenterally, or other route. CIFN and ribavirin may be administered by the same route of administration or by different routes of administration. The therapeutic agents can be administered by any suitable means including, but not limited to, for example, oral, rectal, nasal, topical (including transdermal, aerosol, buccal and sublingual), vaginal, parenteral (including subcutaneous, intramuscular, intravenous and intradermal), intravesical or injection into an affected organ.

The therapeutic agent(s) may administered in a unit dosage form and may be prepared by any methods well known in the art. Such methods include combining the compounds of the present invention with a pharmaceutically acceptable carrier or diluent which constitutes one or more accessory ingredients. A pharmaceutically acceptable carrier is selected on the basis of the chosen route of administration and standard pharmaceutical practice. Each carrier must be “pharmaceutically acceptable” in the sense of being compatible with the other ingredients of the formulation and not injurious to the subject. This carrier can be a solid or liquid and the type is generally chosen based on the type of administration being used.

Examples of suitable solied carriers include lactose, sucrose, gelatin, agar ana bulk powders. Examples of suitable liquid carriers include water, pharmaceutically acceptable fats and oils, alcohols or other organic solvents, including esters, emulsions, syrups or elixirs, suspensions, solutions and/or suspensions, and solution and or suspensions reconstituted from non-effervescent granules and effervescent preparations reconstituted from effervescent granules. Such liquid carriers may contain, for example, suitable solvents, preservatives, emulsifying agents, suspending agents, diluents, sweeteners, thickeners, and melting agents. Preferred carriers are edible oils, for example, corn or canola oils. Polyethylene glycols, e.g. PEG, are also good carriers.

Any drug delivery device or system that provides for the dosing regimen of the instant invention can be used. A wide variety of delivery devices and systems are known to those skilled in the art.

### Determining Effectiveness of Treatment

Whether a subject method is effective in treating an HCV infection can be determined by measuring viral load, or by measuring a parameter associated with HCV infection, including, but not limited to, liver fibrosis.

Viral load can be measured by measuring the titer or level of virus in serum. These methods include, but are not limited to, a quantitative polymerase chain reaction (PCR) and a branched DNA (bDNA) test. Quantitative assays for measuring the viral load (titer), of HCV RNA have been developed. Many such assays are available commercially, including a quantitative reverse transcription PCR (RT-PCR) (Amplicor HCV Monitor™, Roche Molecular Systems, New Jersey); and a branched DNA (deoxyribonucleic acid) signal amplification assay (Quantiplex™ HCV RNA Assay (bDNA), Chiron Corp., Emeryville, Calif.). See, e.g., Gretch et al. (1995) Ann. Intern. Med. 123:321-329.

Another method of determining viral load is by measuring the level of serum antibody to HCV. Methods of measuring serum antibody to HCV are standard in the art and include enzyme immunoassays, and recombinant immunoblot assays, both of which involve detection of antibody to HCV by contacting a serum sample with one or more HCV antigens, and detecting any antibody binding to the HCV antigens using an enzyme labeled secondary antibody (e.g., goat anti-human IgG). See, e.g., Weiss et al. (1995) Mayo Clin. Proc. 70:296-297; and Gretch (1997) Hepatology 26:43S-47S.

While viral titers are the most important indicators of effectiveness of a dosing regimen, other parameters can also be measured as secondary indications of effectiveness. Secondary parameters include reduction of liver fibrosis; and reduction in serum levels of particular proteins, as described below.

Liver fibrosis reduction is determined by analyzing a liver biopsy sample. An analysis of a liver biopsy comprises assessments of two major components: necroinflammation assessed by “grade” as a measure of the severity and ongoing disease activity, and the lesions of fibrosis and parenchymal or vascular remodeling as assessed by “stage” as being reflective of long-term disease progression. See, e.g., Brunt (2000) Hepatol. 31:241-246; and METAVIR (1994) Hepatology 20:15-20. Based on analysis of the liver biopsy, a score is assigned. A number of standardized scoring systems exist which provide a quantitative assessment of the degree and severity of fibrosis. These include the METAVIR, Knodell, Scheuer, Ludwig, and Ishak scoring systems.

Serum markers of liver fibrosis can also be measured as an indication of the efficacy of a subject treatment method. Serum markers of liver fibrosis include, but are not limited to, hyaluronate, N-terminal procollagen III peptide, 7S domain of type IV collagen, C-terminal procollagen I peptide, and laminin. Additional biochemical markers of liver fibrosis include α-2-macroglobulin, haptoglobin, gamma globulin, apolipoprotein A, and gamma glutamyl transpeptidase.

Another secondary indicator of effectiveness of a treatment regimen is serum levels of serum alanine aminotransferase (ALT). Serum ALT levels are measured, using standard assays. In general, an ALT level of less than about 80, less than about 60, less than about 50, or about 40 international units per liter of serum is considered normal. In some embodiments, an effective amount of IFNα is an amount effective to reduce ALT levels to less than about 200 IU, less than about 150 IU, less than about 125 IU, less than about 100 IU, less than about 90 IU, less than about 80 IU, less than about 60 IU, or less than about 40 IU.

Subjects Suitable for Treatment

Individuals who have been clinically diagnosed as infected with HCV are suitable for treatment with the methods of the instant invention. Individuals who are infected with HCV are identified as having HCV RNA in their blood, and/or having anti-HCV antibody in their serum. Such individuals include anti-HCV ELISA-positive individuals, and individuals with a positive recombinant immunoblot assay (RIBA). Such individuals may also, but need not, have elevated serum ALT levels.

Patients for whom the therapy of the invention is of particular benefit include treatment failure patients, which include patients who failed to respond to previous HCV therapy (referred to as “non-responders”) or who initially responded to previous therapy, but in whom the therapeutic response was not maintained (referred to as “relapsers”). The previous therapy generally can include treatment with IFN-α monotherapy or IFN-α combination therapy, where the combination therapy may include administration of IFN-α and an antiviral agent such as ribavirin. As non-limiting examples, individuals may have an HCV titer of at least about 10⁵, at least about 5×10⁵, or at least about 10⁶, genome copies of HCV per milliliter of serum.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method for treating a hepatitis C virus infection in an individual, the method comprising administering consensus interferon-α (CIFN) and ribavirin, wherein CIFN is administered in a therapeutic regimen comprising a first dosing regimen of CIFN, followed by a second dosing regimen of CIFN, wherein the lowest average daily serum concentration of CIFN achieved by the first dosing regimen is greater than the highest average daily serum concentration of CIFN achieved by the second dosing regimen, and wherein the individual treated has failed previous IFN-α-based therapy other than CIFN therapy.

2. The method of claim 1, wherein ribavirin is administered during administration of at least the last dosing event of the second dosing regimen.

3. The method of claim 2, wherein ribavirin is administered with additional dosing events continuous with the last dosing event during which ribavirin is administered.

4. The method of any one of claims 1-3, wherein the individual failed to respond to previous IFN-α-based therapy other than CIFN therapy.

5. The method of any one of claims 1-3, wherein the individual has suffered a relapse following cessation of IFN-α therapy other than CIFN therapy.

6. The method of claim 1, wherein the first dosing regimen comprises administering 15 μg CIFN per day for eight weeks, wherein the second dosing regimen comprises administering 9 μg CIFN per day for a period of 16 to 40 weeks, and wherein ribavirin is administered at 1000 to 1200 mg per day throughout the therapeutic regimen.

7. The method of claim 1, wherein the first dosing regimen comprises administering 15 μg CIFN per day for eight weeks, wherein the second dosing regimen comprises administering 9 μg CIFN three times per week for 16 to 40 weeks, and wherein ribavirin is administered at 1000 to 1200 mg per day throughout the therapeutic regimen.

8. The method of claim 1, wherein the first dosing regimen comprises administering 15 μg CIFN per day for eight weeks, wherein the second dosing regimen comprises administering 15 μg; CIFN three times per week for 16 to 40 weeks, and wherein ribavirin is administered at 1000 to 1200 mg per day throughout the therapeutic regimen.

9. The method of claim 1, wherein the first dosing regimen comprises administering 15 μg CIFN per day for eight weeks, wherein the second dosing regimen comprises administering 15 μg CIFN three times per week for 16 to 40 weeks, and wherein ribavirin is administered at 1000 to 1200 mg per day during the second dosing regimen.

10. The method of claim 1, wherein the first dosing regimen comprises administering 27 μg CIFN per day for four weeks followed by administering 18 μg CIFN per day for eight weeks, wherein the second dosing regimen comprises administering 9 μg CIFN per day for 12 weeks followed by administering 9 μg CIFN three times per week for 24 weeks, and wherein ribavirin is administered at 1000 to 1200 mg per day throughout the therapeutic regimen.

11. The method of claim 1, wherein the first dosing regimen comprises administering 27 μg CIFN per day for four weeks followed by administering 18 μg CIFN per day for eight weeks, wherein the second dosing regimen comprises administering 9 μg CIFN per day for 12 weeks followed by administering 9 μg CIFN three times per week for 24 weeks, and wherein ribavirin is administered at 1000 to 1200 mg per day beginning with the eight-week course of 18 μg CIFN per day and continuing ribavirin administration for the remainder of the therapeutic regimen.

12. The method of claim 1, wherein the first dosing regimen comprises administering 27 μg CIFN per day for four weeks followed by administering 18 μg CIFN per day for eight weeks, wherein the second dosing regimen comprises administering 9 μg CIFN per day for 12 weeks followed by administering 9 μg CIFN three times per week for 24 weeks, and wherein ribavirin is administered at 1000 to 1200 mg per day beginning with the 12-week course of 9 μg CIFN per day and continuing ribavirin administration for the remainder of the therapeutic regimen.

13. The method of claim 1, wherein the first dosing regimen comprises administering 27 μg CIFN per day for four weeks followed by administering 18 μg CIFN per day for eight weeks, wherein the second dosing regimen comprises administering 9 μg CIFN per day for 12 weeks followed by administering 9 μg CIFN three times per week for 24 weeks, and wherein ribavirin is administered at 1000 to 1200 mg per day beginning with the 24-week course of 9 μg CIFN three times per week and continuing ribavirin administration for the remainder of the therapeutic regimen.

14. The method of claim 1, wherein the first dosing regimen comprises administering 18 μg CIFN per day for four weeks, wherein the second dosing regimen comprises administering 9 μg CIFN per day for 20 weeks followed by administering 9 μg CIFN three times per week for 24 weeks, and wherein ribavirin is administered at 1000 to 1200 mg per day throughout the therapeutic regimen.

15. The method of claim 1, wherein the first dosing regimen comprises administering 18 μg CIFN per day for four weeks, wherein the second dosing regimen comprises administering 9 μg CIFN per day for 20 weeks followed by administering 9 μg CIFN three times per week for 24 weeks, and wherein ribavirin is administered at 1000 to 1200 mg per day beginning with the 20-week course of 9 μg CIFN per day and continuing ribavirin administration for the remainder of the therapeutic regimen.

16. The method of claim 1, wherein the first dosing regimen comprises administering 18 μg CIFN per day for four weeks, wherein the second dosing regimen comprises administering 9 μg CIFN per day for 20 weeks followed by administering 9 μg CIFN three times per week for 24 weeks, and wherein ribavirin is administered at 1000 to 1200 mg per day beginning with the 24-week course of 9 μg CIFN three times per week and continuing ribavirin administration for the remainder of the therapeutic regimen.

17. The method of claim 1, wherein the first dosing regimen comprises administering 9 μg CIFN per day for 8 to 12 weeks, wherein the second dosing regimen comprises administering 9 μg CIFN three times per week for 36 to 40 weeks, and wherein ribavirin is administered at 1000 to 1200 mg per day throughout the therapeutic regimen.

18. The method of claim 1, wherein the first dosing regimen comprises administering 9 μg CIFN per day for 8 to 12 weeks, wherein the second dosing regimen comprises administering 9 μg CIFN three times per week for 36 to 40 weeks, and wherein ribavirin is administered at 1000 to 1200 mg per day throughout the second dosing regimen.

19. The method of claim 1, wherein the therapeutic regimen achieves a sustained viral response.
